(19) United States
(12) Patent Application Publication
(10) Pub. No.: US 2008/0102127 A1
(43) Pub. Date: May 1, 2008

(54) HYBRID LIPID-POLYMER NANOPARTICULATE DELIVERY COMPOSITION

(76) Inventors: Hai Yan Gao, (US); Joseph Schwarz, (US); Michael Weisspapir, (US)

Correspondence Address: ALPHARX INC., 168 KONRAD CRESCENT, SUITE 200, MARKHAM, L3R 9T9, CA

(21) Appl. No.: 11/848,484

(22) Filed: Aug. 31, 2007

Related U.S. Application Data
(60) Provisional application No. 60/854,458, filed on Oct. 26, 2006.

(51) Int. Cl. A61K 31/7034; A61K 31/496; A61K 31/4709; A61K 31/65; A61K 31/56; A61K 9/14

(52) U.S. Cl. 424/489; 514/171; 514/458; 514/253.08; 514/312; 514/152; 514/35; 977/906

(57) ABSTRACT

The invention relates to a nanoparticulate colloidal delivery vehicle comprising a biodegradable polymer in combination with a hydrophobic lipid component. Variation of the lipid and polymer types and variation in the ratio between the polymer and lipid components allows regulation of drug loading and release rate.

### RELATED APPLICATIONS

This application claims priority from filed U.S. Provisional Patent Application Ser. No. 60/854,458, entitled, “Hybrid Lipid-Polymer Nanoparticulate Delivery Composition”, filed Oct. 26, 2006.

### FIELD OF THE INVENTION

The invention relates to a hybrid lipid-polymer nanoparticle, compositions comprising same, and uses thereof, such as in drug delivery.

### BACKGROUND OF INVENTION

Biodegradable or biocompatible polymeric nanoparticles are widely used as delivery systems for different applications: targeted drug delivery, sustained release of incorporated materials, vaccination and immunization, imaging and other applications. [Sahoo et al., 2003, Vauthier C. et al, 2003]

Other types of nanoparticulate colloidal delivery systems, such as liposomes and solid lipid nanoparticles, are also extensively represented in the field of targeted drug delivery. Multiple liposomal and nanoparticulate colloidal formulations either with drugs or with cosmetic agents were successfully used for human applications (e.g., Doxil®, liposomal Doxorubicin; Ambisome® and Abelcet®; colloidal Amphotericin compositions; Diazemuls®, submicron emulsion with Diazepam; Diprivan®, injectable Propofol emulsion, etc.). Solid lipid nanoparticles (SLN) are intensively investigated as vehicles for drug delivery. [Muller R. H. et al., 2004]

Existing types of biocompatible colloidal DDS have some limitations: limited drug loading, visible cytotoxicity of certain polymers, used for nanoparticle manufacturing [Borm P J, et al., 2006; Vauthier C. et al., 2003], poor compatibility with incorporated components and difficulties in regulation or delivery rate from polymeric nanoparticles, low physical stability of SLN and liposomal formulations.

Nanocapsules combine a liquid lipid core coated with a polymeric layer. These particles are suitable for high loading of hydrophobic compounds but are difficult to lyophilize due to very thin polymeric film, protecting the liquid interior. [Abdelwahed W et al., 2006]

Recently, [Wong H. L. et al., 2006] improved anti-cancer efficacy and drug loading for hybrid nanoparticles, prepared from a combination of charged polymers with polymerized polar epoxydized oil and loaded with Doxorubicin was demonstrated. The main limitation factor for such nanoparticles is complexity of polymerization epoxydized soy bean oil and uncertainty of biological activity of such polymeric lipids and their biodagradability.

The article of Mu L and Feng S. describes the incorporation of paclitaxel into polymeric microparticles, modified with combination of dipalmitoylphosphatidylcholine and cholesterol. These particles were prepared by spray drying technique, while the lipid-lecithin combination was used as a surfactant and located on the particle surface.

U.S. Patent Application No. 0060177495 describes polymeric nanoparticles, coated with phospholipid layer made of phosphatidylcholine of pegylated phospholipids. In this case, phospholipids are used as a functional external surfactant to protect nanoparticles from aggregation and modify body distribution, but the polymeric core is not modified.

### SUMMARY OF THE INVENTION

In one aspect, the present invention provides a biocompatible and stable polymer-lipid hybrid nanoparticulate vehicle. In one aspect, the polymer-lipid hybrid nanoparticulate vehicle is stable. In one embodiment, the formulations did not show phase separation, drug leaking or precipitation and serious change in particle size and other physico-chemical parameters during storage through a reasonable shelf life. In one embodiment, the nanoparticles are comprised of a homogenous combination of biodegradable polymer and hydrophobic solid lipid component. In one aspect, all the components are evenly and uniformly mixed forming a solution. In another aspect, the hydrophobic solid lipid component is a water repelling lipid material. In another aspect, the melting point of solid lipid is greater than 25° C. In one embodiment, the lipid is a non-polymerized lipid. The presence of lipid, evenly distributed in the polymeric matrix of a nanoparticle, allows improved incorporation of hydrophobic biologically active compounds compared with nanoparticles built of polymer alone. The polymeric matrix of a nanoparticle is a discontinuous phase representing the main (central) part of the nanoparticle, essentially comprising polymer. Low toxicity of incorporated lipid decreases toxicity of the vehicle and alleviates the regulation of drug release rate. The vehicle helps to administer high doses of biologically active compounds safely and without an unnecessary level of side effects. Since the lipid is solid, the lyophilization process is easy and uncomplicated. Such polymer-lipid hybrid nanoparticles have not been described previously.

It was surprisingly found that solid lipids, such as triglycerides, sterols, waxes, etc. demonstrated behavior different from liquid lipidic compounds. Whereas liquid oils form nanocapsules with liquid cores or highly plasticized soft polymeric aggregates or micelles (e.g., PCL or PLGA with benzyl benzoate) [see Guterres S. et al., 2000], solid lipids mingle with hydrophobic polymers forming nanoparticles with components uniformly distributed throughout the particle. Therefore, polymer-lipid hybrid nanoparticles can better incorporate either non-polar or polar compounds. In one aspect, nanoparticles are prepared using traditional methods for nanoparticle preparation: initial dissolving of drug, polymer and lipid in organic solvent (miscible or not-miscible with water) and then emulsification for co-precipitation with water-surfactant media, followed with solvent elimination. In another aspect, hydrophilic drugs were incorporated after hydrophobization via counter-ion interaction. In one aspect of the invention, hydrophobic counter-ions can better incorporate into lipidic regions of nanoparticles, and the efficiency of entrapment is higher than for polymer-only nanoparticle vehicle.

In another embodiment of the invention, polymer-lipid hybrid nanoparticles could be prepared by any method for nanoparticle manufacturing: emulsification of water-immiscible organic solution, co-precipitation from water miscible solvent, desalting, or other known processes. Different methods of preparation can be used to obtain different particle size distribution, drug loading and inclusion rate, as desired, for suitable nanoparticulate controlled drug delivery.

Other features and advantages of the present invention will become apparent from the following detailed description. It should be understood, however, that the detailed description and the specific examples, while indicating preferred embodiments of the invention, are given by way of illustration only, since various changes and modifications within the spirit and scope of the invention will become apparent to those skilled in the art from reading the detailed description.

### BRIEF DESCRIPTION OF THE DRAWINGS

The invention will now be described in relation to drawings which will illustrate certain embodiments of the invention but are not intended to limit the scope of the invention:

FIG. 1 is a graphical representation showing the release rate of gentamycin from hybrid polymer-lipid nanoparticles, showing the suppression of gentamycin release where lipid has been added to the nanoparticles;

FIG. 2 is a graphical representation showing the release rate of gentamycin from hybrid polymer-lipid nanoparticles, showing the regulation of release rate by lipid variation;

FIG. 3 is a graphical representation showing comparative drug loading into nanoparticles;

FIG. 4 is a graphical representation showing mice survival in E. coli sepsis model with gentamycin;

FIG. 5 is a graphical representation showing mice survival rates in sepsis model with streptomycin; and

FIG. 6 is a schematic diagram illustrating one of the embodiments of the nanoparticle of the invention.

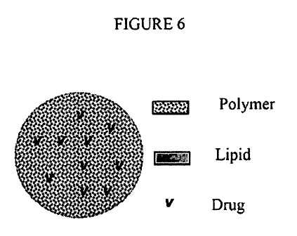

### DETAILED DESCRIPTION OF THE INVENTION

It was unexpectedly found that the combination in nanoparticles of biodegradable polymer with evenly distributed solid lipid provides better drug loading, good stability of the colloidal vehicle and that this makes it possible to regulate release and degradation parameters. Hybrid polymer-lipid nanoparticles (HPLNP) comprise a biodegradable polymer, e.g., polylactic-polyglycolic copolymer (PLGA), polycaprolactone (PCL) and a solid lipid, e.g., tristearin, tripalmitin, glycerin stearate, cholesterol, tocopherol palmitate, tocopheryl succinate, stearyl stearate, tribehenin, cetostearyl alcohol, benzoyl behenate, stearic acid, carnauba or candelilla wax, cocoa butter, Suppocire™ CM/DM, Wecobee™ M and other lipids, having a melting point beyond 20° C., i.e., solid at room temperature.

To prepare HPLNP, the selected polymer, lipid and drug are dissolved in organic solvent, e.g. in water immiscible solvent, such as methylene chloride or ethylacetate for the emulsification method, or in water miscible solvent—e.g., acetone, N-methylpyrrolidone, ethyllactate—for the precipitation method. For charged compounds a hydrophobic counter-ion can be also added to an organic solution, e.g., cholesteryl sulfate of cetylphosphate for amines, gtuanidines and other basic molecules, stearylamine, DMAE-carbamoyl cholesterol or another appropriate amine for acidic substances. The prepared solution is mixed with the water phase and undergoes an emulsification or a precipitation process. Highly water soluble compounds, such as aminoglycosides or other antibiotics, can be preliminarily dissolved in a small amount of water and dispersed in a lipid-polymer solution, containing counter-ion.

To stabilize a colloidal system of HPLNP, different surfactants can be used. Since formulations were proposed for oral or parenteral administration, physiologically acceptable components with low toxicity were involved, such as: polysorbates, ethoxylated castor oil, PEG-1000 tocopherol succinate, phosphatidylcholine (hydrogenated and non-hydrogenated lecithin), sucrose esters, block-copolymers of polyethylene glycol and propylene glycol (Poloxamers), etc.

Biocompatible polymers, used for preparation of HPLNP, were selected to provide reasonable degradation and drug release time in order to reach a desired drug concentration in targeted tissues and organs. Polymers and lipids are soluble one in another, and form a single, homogeneous phase, when combined, at least in some ratios, otherwise, the phases in the nanoparticle matrix separate and the colloidal system becomes unstable. Best results were obtained with glycerides, waxes, cholesterol, cholesterol esters and other sterols, long chain solid fatty acids and alcohols and their esters.

Lipid regions in polymeric matrixes of nanoparticles incorporate hydrophobic drugs with higher efficiency. For example, Ubidecarenone, Prednisolone and Prednisolone acetate, Rifampicin, Doxorubicin, and Cyclosporin were successfully incorporated with high yield and good inclusion level.

HPLNP

In one embodiment, the invention describes a nanoparticulate colloidal delivery vehicle where nanoparticles are composed of water insoluble biocompatible polymer and solid lipid material, uniformly distributed in nanoparticle polymeric matrix, an outer layer, surrounding the particle and comprised of surfactant(s). This layer additionally may comprise phospholipid(s), pegylated phospholipid(s), water soluble or water swellable polymer(s) and targeting/recognizing compounds.

In another embodiment, the nanoparticulate colloidal delivery vehicle may be associated with nanoparticles.

The biocompatible polymer may be selected from polyacrylates, polycyanoacrylates, polylactic acid, polyglycolic acid, lactide-glycolide copolymers, lactide-glycolide-polyethyleneglycol copolymers, polyorthoesters, polyanhydrides, biodegradable block-copolymers, poly(caprolactone), poly(butyrolactone), poly(valerolactone) and other polylactones and their copolymers.

In one embodiment, the lipid is solid at room temperature. In one embodiment, the lipid melts at a temperature higher than 25° C.

In one embodiment, the solid lipid may be one or more natural or synthetic lipids, fats, mono-, di- and triglycerides, fatty acids, fatty alcohols, waxes, cholesterol and cholesterol derivatives, aliphatic and aromatic esters.

In one embodiment, the polymer and lipid are in a ratio sufficient to maintain homogenous polymer association with said lipid and pharmaceutically active compound.

In one embodiment, the polymer/lipid weight ratio is between 99:1 and 1:99.

In another embodiment, the polymer/lipid weight ration is between 10:1 and 1:10.

In one embodiment, the lipid may be one or more of glyceride of saturated fatty acid with chain length from 12 to 30 carbons, glycerol monostearate, glycerol distearate, glycerol tristearate, glycerol stearates, cholesterol, cholesterol ester, aliphatic ester, aromatic ester, or tocopheryl ester.

In one embodiment, the tocopheryl ester may be tocopheryl succinate or tocopheryl palmitate.

In another embodiment, the nanoparticulate colloidal delivery vehicle may further include surfactants, stabilizers, rheology modifiers, antioxidants and preservatives.

In another embodiment, the surfactants may be selected from anionic, cationic, non-ionic or amphoteric type surfactants.

In another embodiment, the nanoparticulate colloidal delivery vehicle may further comprise a pharmaceutically active compound.

In another embodiment, the pharmaceutically active compound may be an antibiotic, anti-neoplastic agent, steroidal hormone, sex hormone, peptide, non-steroidal anti-inflammatory drug (NSAID), antifungal drug, anti-viral drug, neuraminidase inhibitor, opioid agonist or antagonist, calcium channel blocker, antiangiogenic drug, diagnostic compound or vaccine.

In another embodiment, where the pharmaceutically active ingredient is an antibiotic, the antibiotic may be selected from the group of aminoglycosides (Gentamycin, Tobramycin, Streptomycin, Amikacin), macrolides (Azithromycin, Clarithromycin), Rifampines (Rifampicin, Rifabutine), fluoroquinolones (Ciprofloxacin, Moxifloxacin, Gatifloxacin), Tetracyclines (Doxicyclin, Minocyclin).

In another embodiment, where the pharmaceutically active ingredient is a steroidal hormone, the steroid hormone may be selected from the group of corticosteroidal hormones (Hydrocortisone, Progesterone, Prednisolone, Betamethasone, Dexamethasone, fluorinated corticosteroids), anabolic steroids (Retabolil, Nerobolil, Androstenolone, Androstenone, Nandrolol), physiologically equivalent hormones derivatives or combinations thereof.

In another embodiment, where the pharmaceutically active ingredient is a hormone antagonist, the hormone antagonist may be selected from group of anti estrogens (Tamoxifen, Raloxifen), LHRH (luteinizing hormone-releasing hormone) antagonist (Leuprolide, Goserelin), gonadotropin-releasing hormone (GnRH) antagonist (Cetrorelix, Ganirelix).

In another embodiment, where the pharmaceutically active ingredient is a sex hormone, the sex hormone may be selected from the group consisting of androgens (testosterone, dihydrotestosterone) and estrogens (estradiol, norestradiol), physiologically equivalent hormones derivatives or combinations thereof.

In another embodiment, where the pharmaceutically active ingredient is an anti-neoplastic agent, the anti-neoplastic agent may be selected from anticancer antibiotics (Doxorubicin, Daunorubicin, Valrubicin, Bleomycin, Dactinomycin, Epirubicin, Idarubicin, Mitoxantrone, Mitomycin), Topoisomerase inhibitors (Topotecan, Irinotecan), plant alkaloids and their derivatives (Paclitaxel, Docetaxel, Etoposide, Camptothecin, Vinblastine, Vincristine, Vindesine, Vinorelbine), aromatase inhibitors (Anastrozole, Letrozole), antimetabolites (Methotrexate, Pemetrexed, Raltitrexed, Cladribine, Clofarabine, Fludarabine, Mercaptopurine, Tioguanine, Capecitabine, Cytarabine, Fluorouracil, Gemcitabine).

In one embodiment, the nanoparticulate colloidal delivery vehicle may be associated with a pharmaceutical composition.

In one embodiment, an effective or therapeutically effective amount of the composition is administered. An “effective amount” as used herein means an amount effective, at dosages and for periods of time necessary to achieve the desired results. Administration of a therapeutically effective amount of pharmaceutical compositions of the present invention is defined as an amount effective, at dosages and for periods of time necessary to achieve the desired therapeutic result. For example, an effective or therapeutically effective amount of a substance may vary according to factors such as the disease state, age, sex, and weight of the individual, and the ability of the substance to elicit a desired response in the individual. Dosage regimes may be adjusted to provide the optimum response. For example, several divided doses may be administered daily or the dose may be proportionally reduced as indicated by the exigencies of the therapeutic situation. In addition, an individual skilled in the art will appreciate that various excipients such as those set out in Remington: The Science and Practice of Pharmacy, 20^{th} edition, [Remington, 2000] may be added to the end composition.

The present invention is described in reference to the following Examples, which are set forth to aid in the understanding of the invention, and should not be construed to limit in any way the scope of the invention.

### EXAMPLES

A number of HPLNP were prepared in accordance with the invention as listed in Table 1.

Prepared by Emulsification Process

Emulsification process: Cholesterol (50 mg) and Polycaprolactone (450 mg, mw 10,000) were dissolved in 10 ml of Ethylacetate, saturated with water. Prednisolone (50 mg) was added to obtained solution and stirred until completely dissolved. Solution in organic solvent was added to 40 ml of 3% Tween-80 (Polysorbate 80) in purified water, sonicated 60 seconds using ultrasonic processor at 120 watt and homogenized using high pressure homogenizer (e.g., Avestin Emulsiflex C-5 or similar) for 5 cycles at 8,000-15,000 psi. After homogenization organic solvent was evaporated under reduced pressure, suspension was concentrated to 20 ml and passed through membrane filter with pore size 0.45 mcm. Particle size was estimated using Malvern Nanosizer Nano-S analyzer in water media. (Examples 1 of Table 1)

Prepared by Precipitation Process

Polymer (polylactic-polyglycolic acid copolymer, Resomer™ 504H, 200 mg), lipid components (Suppocire™, 80 mg, and Tocopherol succinate, 10 mg) and Doxorubicin (20 mg) were dissolved in Acetone (22 ml). Obtained solution was added through syringe (needle with gauge 22) into 50 ml of 1% Tween-80 with intensive stirring. After 30 minutes of stirring, the organic solvent was evaporated at reduced pressure, suspension was concentrated to 20 ml and passed through membrane filter with pore size 0.45 mcm. Particle size was estimated using Malvern Nanosizer Nano-S analyzer in water media. (Example 12 of Table 1)

The association of the included drug with nanoparticles was estimated using centrifugation method (Amicon Ultrafree™ centrifugal filter with cellulose membrane (cutoff 30,000 Dalton) from Millipore Corp.), and drug concentration in filtrate was measured by HPLC.

Nanoparticles, prepared either by homogenization-emulsification or by precipitation method showed good stability and reasonable particle size and size distribution. The solid lipid did not separate from polymeric nanoparticles and is evenly distributed inside the nanoparticles.

Incorporation of Solid Lipids in Polymeric Nanoparticles Regulates Drug Release Rate

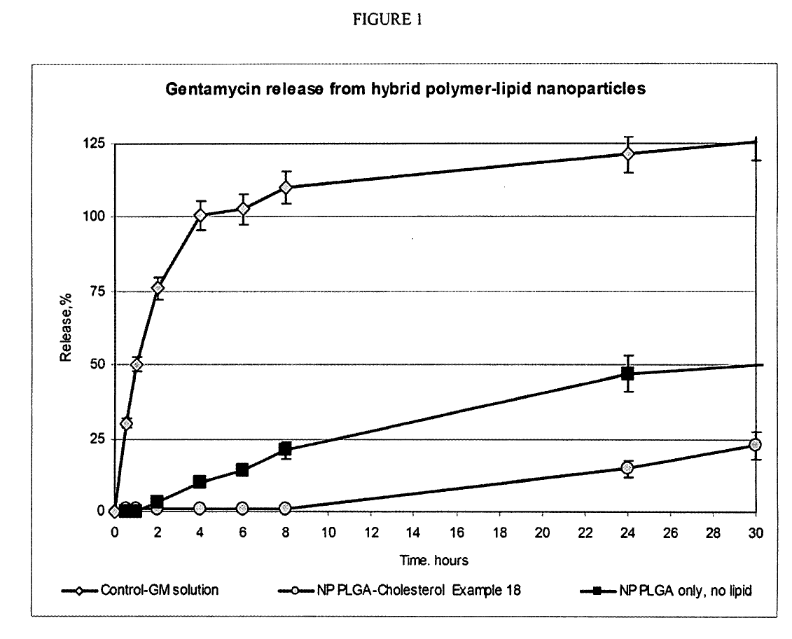

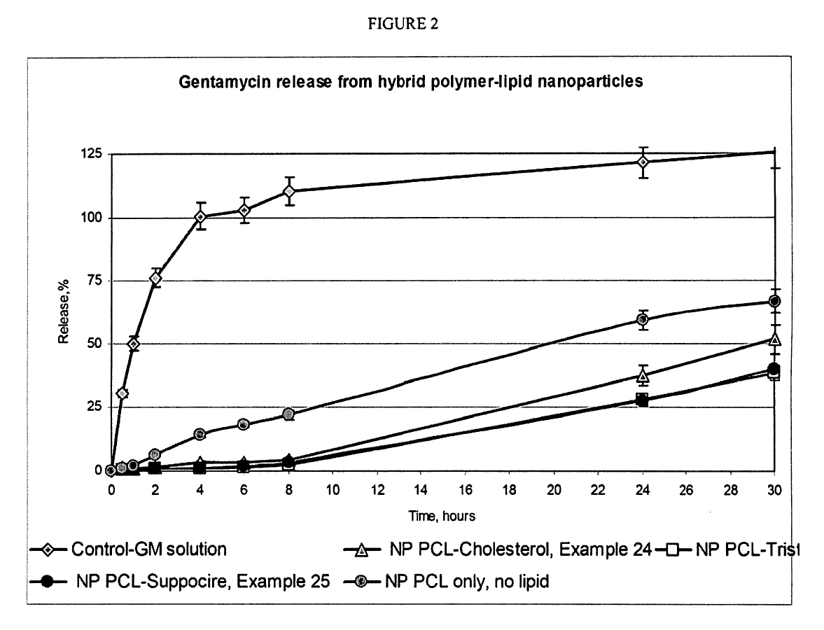

Unexpectedly, it was found that incorporation of solid lipids into polymeric nanoparticles permits regulation of the behavior of the colloidal delivery system. The release rate of the drug incorporated into polymeric nanoparticles together with solid lipid decreases (FIGS. 1 and 2). Such behavior differs from the release of the same drug incorporated into polymeric nanocapsules which contain liquid lipid core; release from nanocapsules is faster than from nanoparticles. Comparative data may be found in V. Ferranti et al., 1999; Miyazaki S., et al., 2003.

Hybrid lipid-polymeric nanoparticles may be prepared from different polymers and lipids with various melting points, polarity and in a wide range of polymer-to-lipid ratio. This permits the development of a delivery system with optimal properties, suitable for different types of biologically active molecules: polar, non-polar, water soluble, peptides and proteins, etc. Selection of appropriate components of the hybrid colloidal system allows regulation of the release rate for incorporated drugs, protects included active components and suppresses hydrolytic or enzymatic degradation of the polymer as far as stability of the entire colloidal system.

Drug Loading into Polymeric Nanoparticles with and without Lipid

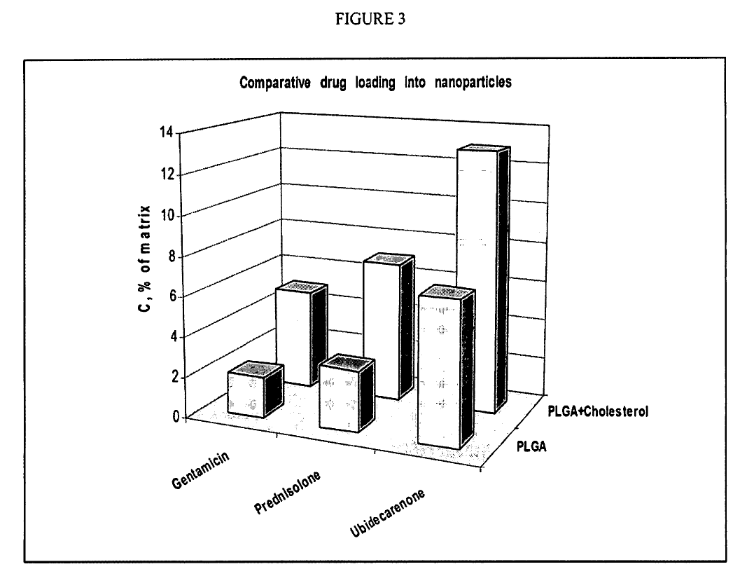

When required, comparative nanoparticles with the same polymers, surfactants and drugs but without lipid were prepared similarly. FIG. 3 represents comparative drug loading into nanoparticles from the same components with and without lipid.

Increase of Drug Efficacy In Vivo of HPLNP

Antibacterial efficacy of antibiotics in hybrid polymer-lipid nanoparticles was evaluated “in vivo” in a sepsis model, induced in mice (BALB/C line) by Escherichia Coli (strain O157). E. Coli O157 strain was chosen as a model infection as one of the most common pathogens that cause nosocomial infections.

Female BALB/c mice 6-8 weeks old were infected intraperitoneally with E. coli (strain O157) at a dose of 2.5×10⁸ cells off E. coli per mouse.

The infected animals were randomized in groups (n=10) and received one of the following formulations:

Streptomycin formulations: 1) Saline; 2) Streptomycin solution in saline, 5 mg/ml 3) Streptomycin in hybrid polymer-lipid nanoparticles (Example 30 in Table 1), 5 mg/ml.

Gentamycin formulations: 1) Saline 2) Gentamycin solution in saline, 2.5 mg/ml; 3) Gentamycin in hybrid polymer-lipid nanoparticles (Example 24 in Table 1), 2.5 mg/ml.

The formulations were injected intraperitoneally (Streptomycin) or intravenously (Gentamycin) in the doses of 1×25 mg/kg (calculated by antibiotic base) 4 h post infection.

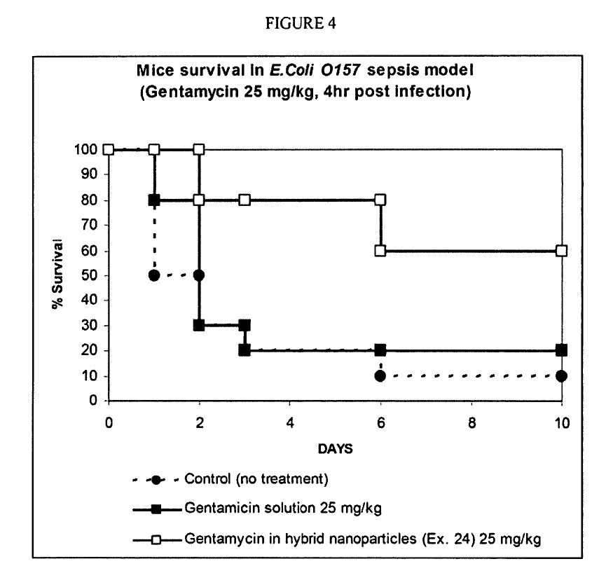

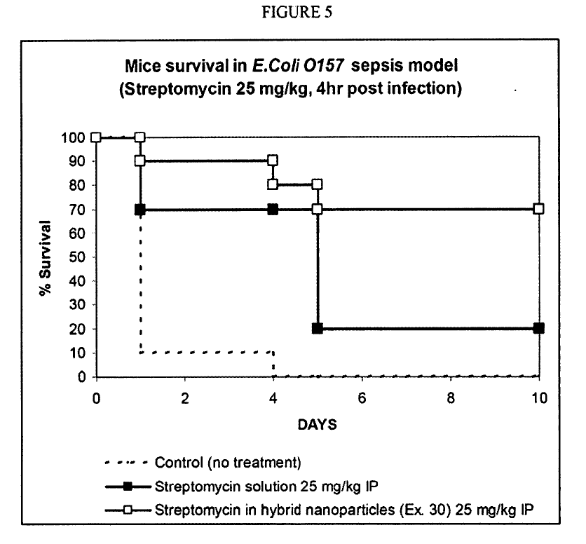

The results of the sepsis treatment are presented in FIGS. 4 and 5. It is clearly visible that the efficacy of Gentamycin was considerably increased when the drug was loaded in the hybrid nanoparticles. A single injection enabled survival of 60% of the animals at Day 10 (FIG. 4), whereas the same dose of the drug in solution was inefficient (20% survived).

A similar effect was obtained for the Streptomycin formulation (see FIG. 5).

While the present invention has been described with reference to what is presently considered to be a preferred embodiment, it is to be understood that the invention is not limited to the disclosed embodiment. To the contrary, the invention is intended to cover various modifications and equivalent arrangements included within the spirit and scope of the appended claims. It should be noted the application is intended to encompass obvious chemical equivalents of the components of the invention as described herein, which are equivalents that produce the same or equivalent desired result for a particular feature of the invention.

All publications, patents, and patent applications are herein incorporated by reference in their entireties, to the same extent as if each individual publication, patent, or patent application was specifically and individually indicated to be incorporated by reference in its entirety.

TABLE 1
Examples of Drug Loaded Hybrid Polymeric-Lipid Nanoparticles

| | | | | | | | | | Size, |
|---|---|---|---|---|---|---|---|---|---|
| Ex. | Drug | Polymer | Lipid | Solvent | Surfactant | Method | Yield | Binding | nm |
| 1 | Prednisolone 50 mg | PCL | Cholesterol | Ethylacetate | Tween-80 3% | Emulsification | 69% | 78.1% | 260 |
| | | 450 mg | 50 mg | 10 ml | | | | | |
| 2 | Prednisolone acetate | PCL | Tristearin 50 mg | Methylene | Poly vinyl alcohol | Emulsification | 81% | 94.6% | 186 |
| | 50 mg | 450 mg | | chloride 8 ml | 1% | | | | |
| 3 | Prednisolone 50 mg | PCL | Cholesterol | Ethylacetate | TPGS 3% | Emulsification | 84% | 80.5% | 77 |
| | | 450 mg | 50 mg | 10 ml | | | | | |
| 4 | Prednisolone 100 mg | PCL | Cholesterol | Ethylacetate | Tween-80 3% | Emulsification | 28% | 71% | 227 |
| | | 100 mg | 50 mg | 10 ml | | | | | |
| 5 | Prednisolone 50 mg | PCL | Cholesterol | Ethylacetate | Tween-80 3% | Emulsification | 56% | 76.6% | 98 |
| | | 100 mg | 50 mg | 10 ml | | | | | |
| 6 | Prednisolone 50 mg | PCL | Tocopheryl | Ethylacetate | Tyloxapol 2.5% | Emulsification | 61% | 91.1% | 134 |
| | | 100 mg | palmitate 50 mg | 10 ml | | | | | |
| 7 | DPH 1 mg | PCL | Cholesterol | Ethylacetate | Tween-80 3% | Emulsification | 100% | 99.8% | 101 |
| | (fluorescent label) | 450 mg | 50 mg | 10 ml | | | | | |
| 8 | Rifampicin 20 mg | PLGA | GMS | Ethylacetate | TPGS 3%, | Emulsification | 86% | 91.4% | 158 |
| | | 200 mg | 200 mg | 10 ml | NaDC 0.5% | | | | |
| 9 | Rifampicin 20 mg | PLGA | Tristearin | Ethylacetate | TPGS 3% | Emulsification | 95% | 89% | 185 |
| | | 200 mg | 200 mg | 10 ml | NaDC 0.5% | | | | |
| 10 | Rifampicin 20 mg | PLGA | GMS | Methylene | Pluronic F68 0.5% | Emulsification | 64% | 13% | 2390 |
| | | 200 mg | 200 mg | chloride 6 ml | | | | | |
| 11 | Rifampicin 20 mg | PLGA | Cholesterol | Ethylacetate | Cremophor EL 3% | Emulsification | 81% | 82.4% | 348 |
| | | 360 mg | 40 mg | 10 ml | | | | | |
| 12 | Rifampicin 20 mg | PLGA | Suppocire 80 mg | Acetone | Tween-80 1% | Precipitation | 61% | 73.4% | 680 |
| | | 200 mg | Tocopherol | 22 ml | | | | | |
| | | | succinate 10 mg | | | | | | |
| 13 | Doxorubicin 10 mg | PLGA | Cholesterol | Methylene | TPGS 1% | Emulsification | 89% | 77.9% | 177 |
| | | 100 mg | 10 mg | chloride 6 ml | NaDC 0.1% | | | | |
| 14 | Doxorubicin 20 mg | PLGA | Cholesterol | Ethylacetate | Cremophor EL 2% | Emulsification | 91% | 72.1% | 201 |
| | | 250 mg | 50 mg | 10 ml | | | | | |
| | | | NaCholSO₄ 12 mg | | | | | | |
| 15 | Gentamycin sulfate | PLGA | GMS | Ethyl acetate | TPGS 3%, | Emulsification | 78% | 68.2% | 78 |
| | 50 mg | 360 mg | 80 mg | 10 ml | NaDC 0.5% | | | | |
| 16 | Gentamycin sulfate | PLGA | Cetostearyl | Methylene | Pluronic F68 0.5% | Emulsification | 66% | 13.5% | 314 |
| | 50 mg | 360 mg | alcohol 40 mg | chloride 6 ml | | | | | |
| 17 | Gentamycin sulfate | PLGA | GMS | Ethylacetate | TPGS 3%, | Emulsification | 79% | 68.6% | 61 |
| | 50 mg | 200 mg | 200 mg | 14 ml | NaDC 0.5% | | | | |
| 18 | Gentamycin sulfate | PLGA | Cholesterol | Ethylacetate | TPGS 3%, | Emulsification | 53% | 51.0% | 55 |
| | 50 mg | 360 mg | 40 mg | 10 ml | NaDC 0.5% | | | | |
| 19 | Gentamycin sulfate | PLGA | Tristearin | Ethylacetate | TPGS 1.5% | Emulsification | 63% | 9.9% | 110 |
| | 50 mg | 360 mg | 40 mg | 10 ml | | | | | |
| 20 | Gentamycin sulfate | PCL | Tristearin | Ethylacetate | Cremophor EL 3% | Emulsification | 80% | 16.6% | 290 |
| | 50 mg | 400 mg | 40 mg | 10 ml | | | | | |
| 21 | Gentamycin sulfate | PLGA | Cholesterol | Ethylacetate | TPGS 3%, | Emulsification | 81% | 85.4% | 13 |
| | 50 mg | 200 mg | 200 mg | 10 ml | NaDC 0.5% | | | | |
| 22 | Gentamycin sulfate | PCL | Suppocire CM | Ethylacetate | TPGS 3%, | Emulsification | 91% | 88.9% | 69 |
| | 50 mg | 360 mg | 40 mg | 10 ml | NaDC 0.5% | | | | |
| 23 | Gentamycin sulfate | PLGA | Tocopherol | Acetone | Tween-80 0.2% | Precipitation | 66% | 21.6% | 194 |
| | 100 mg | 450 mg | succinate 100 mg | 16 ml | | | | | |
| 24 | Gentamycin sulfate | PCL | Cholesterol | Ethylacetate | TPGS 3%, | Emulsification | 84% | 80.9% | 221 |
| | 50 mg | 360 mg | 40 mg | 10 ml | NaDC 0.5% | | | | |
| 25 | Gentamycin sulfate | PCL | Tristearin | Ethylacetate | TPGS 3%, | Emulsification | 88% | 68.1% | 263 |
| | 50 mg | 360 mg | 40 mg | 10 ml | NaDC 0.5% | | | | |
| 26 | Gentamycin sulfate | PLGA | GMS | Ethylacetate | TPGS 3%, | Emulsification | 73% | 60.7% | 80 |
| | 50 mg | 360 mg | 40 mg | 10 ml | NaDC 0.5% | | | | |
| 27 | Vancomycin HCl | PLGA | Cholesterol | Methylene | TPGS 1.5%, | Emulsification | 93% | 96.9% | 172 |
| | 20 mg | 240 mg | 40 mg | chloride 6 ml | NaDC 0.25% | | | | |
| 28 | Streptomycin sulfate | PHB | Cholesteryl | Acetone | Tween-80 2.5% | Precipitation | 44% | 31.9% | 520 |
| | 100 mg | 750 mg | palmitate 40 mg | 25 ml | | | | | |
| | | | Cetyl PO4 16 mg | | | | | | |
| 29 | Streptomycin sulfate | PLGA | GMS 140 mg | Ethylacetate | Tween-80 2.5% | Emulsification | 70% | 54.1% | 281 |
| | 80 mg | 720 mg | Cetyl phosphate | 14 ml | | | | | |
| | | | 12 mg | | | | | | |
| 30 | Streptomycin sulfate | PLGA | Cholesterol | Ethylacetate | Cremophor EL 2% | Emulsification | 86% | 69.3% | 223 |
| | 100 mg | 750 mg | 100 mg | 24 ml | | | | | |
| | | | NaCholSO₄ 25 mg | | | | | | |
| 31 | Streptomycin sulfate | PCL | Tristearin | Ethylacetate | Solutol HS-15 2% | Emulsification | 91% | 81.4% | 289 |
| | 100 mg | 800 mg | 200 mg | 20 ml | NaDC 0.5% | | | | |
| 32 | Paclitaxel 10 mg | PLGA | Cholesterol | Methylene | TPGS 2.5% | Emulsification | 84% | 79.4% | 163 |
| | | 190 mg | 20 mg | chloride 4 ml | | | | | |
| 33 | Etoposide 10 mg | PCL | GMS | Ethylacetate | TPGS 1.5%, | Emulsification | 72% | 89.1% | 348 |
| | | 360 mg | 30 mg | 10 ml | NaDC 0.25% | | | | |
| 34 | Ubidecarenone | PLGA | Tocopheryl | Ethylacetate | Tween-80 2% | Emulsification | 96% | 94.1% | 159 |
| | 100 mg | 400 mg | palmitate 100 mg | 10 ml | | | | | |

Abbreviations in the table:
PCL—Poly (caprolactone), M_{w} = 14,000, Mn = 10,000 (Aldrich)
PLGA—Lactic-Glycolic acid copolymer (Resomer ®, M_{w} 5,000-100,000; Boehringer Ingelheim, Germany)
PHB—Poly-(3-hydroxybutyric) acid (Tₘ 172° C., Aldrich)
GMS—Glyceryl monostearate (Geleol ™ GMS, Gattefosse, France)
Tristearin—Glycerin tristearate, Precirol ™ ATO5 (Gattefosse, France)
NaDC—Sodium Deoxycholate
TPGS—Tocopherol PEG-1000 succinate ester
NaCholSO₄—Cholesteryl sulfate, sodium salt
Cremophor ® EL—Ethoxylated (35) castor oil, BASF
Solutol ® HS-15—Polyoxyl (15) hydroxystearic acid, BASF
Tween ®-80—Polysorbate 20 NF
Pluronic ™ F-68—Poloxamer 188 NF, Polyethylene oxide-polypropylene oxide triple block copolymer

### REFERENCES

US Patent

1. US Patent Application 20060177495 Allen C. et al. “Polymer-lipid delivery vehicles”
   Articles
1. Sahoo S. and Labhasetwar V., Nanotech approaches to drug delivery and imaging; Drug Discov. Today, 2003 Dec. 15; 8(24): 1112-20.
2. Muller R. H., Keck C. M. “Challenges and solutions for the delivery of biotech drugs—a review of drug nanocrystal technology and lipid nanoparticles” Journal of Biotechnology, 2004, No. 113 pp. 151-170
3. Borm P. J, et al., The potential risks of nanomaterials: a review carried out for ECETOC. Particles and Fibre Toxicol., 2006, No. 3 vol. 1 p. 11;
4. Vauthier C. et al., Poly(alkylcyanoacrylates) as biodegradable materials for biomedical applications. Adv. Drug Deliv. Rev., 2003, No. 55 vol. 4 pp. 519-548
5. Abdelwahed W et al., Investigation of nanocapsules stabilization by amorphous excipients during freeze-drying and storage. Eur J Pharm Biopharm., 2006 No. 63 vol. 2 pp. 87-94.
6. Wong H. L. et al., “A New Polymer-Lipid Hybrid Nanoparticle System Increases Cytotoxicity of Doxorubicin Against Multidrug-Resistant Human Breast Cancer Cells” Pharmaceutical Research”, 2006 Vol. 23, No. 7
7. Mu L., Feng S. S. “Fabrication, characterization and in vitro release of paclitaxel (Taxol®) loaded poly (lactic-co-glycolic acid) microspheres prepared by spray drying technique with lipid/cholesterol emulsifiers” Journal of Controlled Release, 2001, No. 76, pp. 239-254
8. Guterres S. et al., “Influence of Benzyl Benzoate as Oil Core on the Physicochemical Properties of Spray-Dried Powders from Polymeric Nanocapsules Containing Indomethacin”, Drug Delivery, 2000, No. 7, pp. 195-199
9. Ferranti V. et al., “Primidone-loaded poly-o-caprolactone nanocapsules: incorporation efficiency and in vitro release profiles”. International Journal of Pharmaceutics, 1999, No. 193, pp. 107-111;
10. Miyazaki S. et al., Poly n-butylcyanoacrylate (PNBCA) nanocapsules as a carrier for NSAIDs: in vitro release and in vivo skin penetration. J Pharm Pharmaceut Sci., 2003, No. 6 vol. 2, pp. 240-245,

We claim:

1. A nanoparticulate colloidal delivery vehicle where nanoparticles are composed of
a) water insoluble biocompatible polymer and
b) solid lipid material, uniformly distributed in nanoparticle polymeric matrix
c) an outer layer, surrounding the particle and comprising of surfactant(s), said layer additionally may comprise phospholipid(s), pegylated phospholipid(s), water soluble or water swellable polymer(s) and targeting/recognizing compounds.

2. A nanoparticulate vehicle of claim 1 further comprise at least one pharmaceutically active compound.

3. A nanoparticulate vehicle of claim 1 wherein said biocompatible polymer is selected from polyacrylates, polycyanoacrylates, polylactic acid, polyglycolic acid, lactide-glycolide copolymers, lactide-glycolide-polyethyleneglycol copolymers, polyorthoesters, polyanhydrides, biodegradable block-copolymers, poly(caprolactone), poly(butyrolactone), poly(valerolactone) and other polylactones and their copolymers.

4. A nanoparticulate vehicle of claim 1 wherein sail solid lipid is not a phospholipid and is selected from natural or synthetic lipids, fats, mono-, di- and triglycerides, fatty acids, fatty alcohols, waxes, cholesterol and cholesterol derivatives, aliphatic and aromatic esters.

5. A nanoparticulate vehicle of claim 4 wherein said lipid is solid at room temperature and melts at temperature higher than 25° C.

6. A nanoparticulate vehicle of claim 4 wherein polymer and lipid are in a ratio sufficient to maintain homogenous polymer association with said lipid and pharmaceutically active compound.

7. A nanoparticulate vehicle of claim 4 wherein said lipid is glyceride of saturated fatty acid with chain length from 12 to 30 carbons.

8. A nanoparticulate vehicle of claim 4 wherein said lipid is glycerol monostearate.

9. A nanoparticulate vehicle of claim 4 wherein said lipid is glycerol distearate.

10. A nanoparticulate vehicle of claim 4 wherein said lipid is glycerol tristearate.

11. A nanoparticulate vehicle of claim 4 wherein said lipid is mixture of glycerol stearates.

12. A nanoparticulate vehicle of claim 4 wherein said lipid is cholesterol.

13. A nanoparticulate vehicle of claim 4 wherein said lipid is cholesterol ester.

14. A nanoparticulate vehicle of claim 4 wherein said lipid is aliphatic ester.

15. A nanoparticulate vehicle of claim 4 wherein said lipid is aromatic ester.

16. A nanoparticulate vehicle of claim 4 wherein said lipid is tocopheryl ester.

17. A nanoparticulate vehicle of claim 16 wherein said tocopheryl ester is tocopheryl succinate.

18. A nanoparticulate vehicle of claim 16 wherein said tocopheryl ester is tocopheryl palmitate.

19. A nanoparticulate vehicle of claim 1 which may further contain surfactants, stabilizers, rheology modifiers, antioxidants and preservatives.

20. A nanoparticulate vehicle of claim 6 wherein the polymer/lipid weight ratio is from about 10:1 to about 1:10.

21. A nanoparticulate vehicle of claim 19 wherein said surfactants are selected from anionic, cationic, non-ionic or amphoteric type surfactants.

22. A nanoparticulate vehicle of claim 2 wherein said pharmaceutically active compound is antibiotic, anti-neoplastic agent, steroidal hormone, sex hormone, peptide, non-steroidal anti-inflammatory drug (NSAID), antifungal drug, anti-viral drug, neuraminidase inhibitor, opioid agonist or antagonist, calcium channel blocker, antiangiogenic drug, diagnostic compound or vaccine.

23. A nanoparticulate vehicle of claim 22 wherein said antibiotic is selected from the group of aminoglycosides (Gentamycin, Tobramycin, Streptomycin, Amikacin), macrolides (Azithromycin, Clarithromycin), Rifampines (Rifampicin, Rifabutine), fluoroquinolones (Ciprofloxacin, Moxifloxacin, Gatifloxacin), Tetracyclines (Doxicyclin, Minocyclin).

24. A nanoparticulate vehicle of claim 22 wherein said steroidal hormone is selected from the group of corticosteroidal hormones (Hydrocortisone, Progesterone, Prednisolone, Betamethasone, Dexamethasone, fluorinated corticosteroids), anabolic steroids (Retabolil, Nerobolil, Androstenolone, Androstenone, Nandrolol), physiologically equivalent hormones derivatives or combinations thereof.

25. A nanoparticulate vehicle of claim 22 wherein said hormone antagonist is selected from group of anti-estrogens (Tamoxifen, Raloxifen), LHRH (luteinizing hormone-releasing hormone) antagonist (Leuprolide, Goserelin), gonadotropin-releasing hormone (GnRH) antagonist (Cetrorelix, Ganirelix).

26. A nanoparticulate vehicle of claim 22 wherein said sex hormone is selected from group of androgens (testosterone, dihydrotestosterone) and estrogens (estradiol, norestradiol), physiologically equivalent hormones derivatives or combinations thereof.

27. A nanoparticulate vehicle of claim 22 wherein said anti-neoplastic agent is selected from anticancer antibiotics (Doxorubicin, Daunorubicin, Valrubicin, Bleomycin, Dactinomycin, Epirubicin, Idarubicin, Mitoxantrone, Mitomycin), Topoisomerase inhibitors (Topotecan, Irinotecan), plant alkaloids and their derivatives (Paclitaxel, Docetaxel, Etoposide, Camptothecin, Vinblastine, Vincristine, Vindesine, Vinorelbine), aromatase inhibitors (Anastrozole, Letrozole), antimetabolites (Methotrexate, Pemetrexed, Raltitrexed, Cladribine, Clofarabine, Fludarabine, Mercaptopurine, Tioguanine, Capecitabine, Cytarabine, Fluorouracil, Gemcitabine).

28. A nanoparticulate vehicle of claim 2 wherein said active compound is predominantly associated with nanoparticles.
